(19) United States
(12) Patent Application Publication
(10) Pub. No.: US 2026/0159616 A1
(43) Pub. Date: Jun. 11, 2026

(54) POUR POINT DEPRESSANT POLYMER FOR MIDDLE DISTILLATE HYDROCARBONS AND PROCESS OF PREPARATION THEREOF

(71) Applicant: HINDUSTAN PETROLEUM CORPORATION LIMITED, Bangalore (IN)

(72) Inventors: Yadagiri Gopalakrishna Tullimilli, Bengaluru (IN); Nilesh Kottari, Bengaluru (IN); Siva Kesava Raju Chinthalapati, Bengaluru (IN); Srinivasa Narasimha Sheshachala, Bengaluru (IN)

(21) Appl. No.: 19/325,997

(22) Filed: Sep. 11, 2025

(30) Foreign Application Priority Data
Dec. 10, 2024 (IN) 202441097280

(51) Int. Cl. C08F 20/54

(52) U.S. Cl. CPC C08F 20/54

(57) ABSTRACT

The present disclosure provides a scalable pour point depressant polymer of poly(alkyl acrylamide)ₘ (alkyl carboxy ethyl acrylate)ₙ copolymer to meet the various grades of specialty middle distillate hydrocarbon pour point specification. Further, the disclosure provides a process for the preparation of copolymer pour point depressant (PPD).

### CROSS REFERENCE TO RELATED APPLICATION

This application claims priority of Indian Application No. 202441097280, filed Dec. 10, 2024, which is hereby incorporated by reference in its entirety.

### TECHNICAL FIELD OF THE INVENTION

The present invention relates to a pour point depressant (PPD) polymer for middle distillate hydrocarbons. Particularly, the present invention provides a novel pour point depressant polymer of poly (alkyl acrylamide)ₘ (alkyl carboxy ethyl acrylate)ₙ copolymer to meet the winter and specialty middle distillate hydrocarbons pour point specification. Further, the invention also provides a process for the preparation of copolymer pour point depressant (PPD).

### BACKGROUND OF THE INVENTION

Middle Distillate is a complex hydrocarbon mixture composed of C₈-C₂₈ hydrocarbons containing n-alkanes or linear alkanes, iso-paraffins, napthenes and aromatics. At low temperatures, the solubility of normal (i.e. linear) alkanes in diesel is reduced, further to waxy phase separation and crystallize Wax crystallization usually occurs in three stages, namely, (i) nucleation, (ii) growth, and (iii) agglomeration. Generally, in stage (i) at low temperature, wax crystals are precipitated below the thermodynamic solubility of wax. In stage (ii), the size of the wax crystal increases, resulting in the change of crystal morphologies such as flakes, needles, and irregular crystals. Stage (iii) marks the agglomeration of wax crystals. Crystallization causes difficulties in the storage, transportation, and disposal of diesel and even billions of dollars in economic losses worldwide every year.

At present, many methods have been developed to improve the low-temperature fluidity of middle distillate mainly in diesel range hydrocarbons. Adding pour point depressants (PPDs) has become the preferred method to improve the low-temperature fluidity of diesel due to its low cost, low dosage requirements, and ease of operation. PPDs, known as cold flow improvers (CFI), are additives that can significantly reduce the pour point (PP) and cold filter-plugging point (CFPP) of diesel fuel and improve the fluidity of diesel fuel at low temperatures. PPDs modify the size and shape of crystals through nucleation, adsorption, or co-crystallization, and inhibit the formation of large wax crystal lattices. They typically have a wax-like paraffinic part that eutectics with wax and a polar component that interferes with the accumulation of wax crystals.

Poly (alkyl methacrylate) (PAMA) with such characteristics has been widely used. PAMA-type PPDs show good sensitivity to various diesel fuels as discussed in patent U.S. Pat. No. 10,072,115B2. PAMA-type PPDs not only lower the PP of diesel fuel but also improves the viscosity index (VI). However, the effect of the PPD on the low-temperature flow properties of diesel fuel is largely affected by the average carbon number, the relative molecular mass of the polymer, and the structure of the diesel fuel. Ethylene-vinyl acetate (EVA) modified maleic anhydride, and PAMA copolymers are the most widely used building blocks of PPDs as discussed in patent U.S. Pat. No. 10,072,115B2. Some of the prior arts discussing the polymeric compositions as pour point depressants have been listed below.

U.S. Pat. No. 10,072,115B2 discloses polymeric compositions obtainable by free-radical polymerization of at least two different alkyl (meth)acrylates in the presence of at least one ethylene-vinyl ester copolymer, the alkyl (meth)acrylates used being a mixture comprising alkyl (meth)acrylates having linear C12- to C60-alkyl radicals and different alkyl (meth)acrylates having linear C1- to C11-alkyl radicals and/or branched C4- to C60-alkyl radicals and/or cyclic C6- to C20-alkyl radicals. The use of such polymeric compositions as pour point depressants for crude oils, mineral oils or mineral oil products.

U.S. Pat. No. 4,956,111A discloses a pour point depressant for lubricating oils comprises a poly(methacrylate) polymer having the repeating unit ##STR1 ##

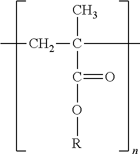

wherein R is an alkyl group having an average chain length in the polymer of 12.6 to 13.8, and n is an integer indicating the number of repeating units, the value of n being sufficient to provide a molecular weight of 10,000 to 300,000 for the polymer, the pour point depressant having the capacity to reduce the stable pour point to −35° C., while being compatible with other additives such as viscosity index improvers and detergents.

Hualin Lin et al., in the paper titled “Research on combined-pour point depressant of methacrylate-acrylamide copolymers and ethylene-vinyl acetate copolymers for diesel fuel” published in Fuel, ISSN 0016-2361, Volume 290, 2021 discloses the combined-pour point depressants (PPDs) potential for the improvement of the cold flow properties of diesel. In this study, free radical polymerization was used to polymerize methacrylate (RMC) and acrylamide (AM) in different molar ratios. Methacrylate-acrylamide copolymers (RMC-AM) were synthesized and the effect of different chain lengths and different monomer molar ratios on the solid point (SP) and cold filter-plugging point (CFPP) of diesel were explored. Then, ethylene-vinyl acetate copolymer (EVA) materials with different VA content were introduced in this study. In addition, the crystallization behaviour and crystal morphology of the treated diesel fuel were discussed. The results showed that the combined-PPD made the wax crystals dissolve in the diesel system. The wax crystals became more uniform and denser under the action of AM polar groups.

In the available prior art, some of the PPDs which were developed had been used for flow assurance to prevent the formation of waxy crystals but some of the problems of crude oil such as plug formation, wax formation, asphaltene formation and reduced flow have not been sufficiently resolved.

### OBJECTIVE OF THE INVENTION

The main object of the present invention is to provide a novel and easily scalable PPD polymer to improve the low-temperature fluidity of middle distillate hydrocarbons.

It is another object of the present invention to provide a process for the preparation of PPD polymer.

It is a yet another object of the present invention to provide a PPD polymer that is cost effective.

It is a yet another object of the present invention to provide a PPD polymer that has significant lower dosage requirements.

### SUMMARY OF THE INVENTION

In one of the embodiments, the present invention provides a pour point depressant polymer, wherein the pour point depressant is a poly(alkyl acrylamide)ₘ (alkyl carboxy ethyl acrylate)ₙ copolymer of formula 1:

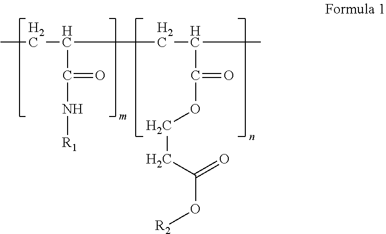

wherein R₁ is selected from H, C1 to C4 alkanes, cyclic alkanes,
wherein R₂ is selected from C₈ to C₂₄ both alkanes and cycloalkanes, preferably alkanes selected from —C₈H₁₇, —C₁₀H₂₁, —C₁₂H₂₅, —C₁₄H₂₉, —C₁₆H₃₃, —C₁₈H₃₇, —C₂₀H₄₁, —C₂₂H₄₅ and —C₂₄H₄₉, and cycloalkanes.
and m is an integer indicating the number of alkyl acrylamide repeating units and n is an integer indicating the number of alkyl carboxy ethyl acrylate repeating units.

In an embodiment of the present invention, the copolymer is prepared by polymerisation of monomers alkyl carboxy ethyl acrylate and alkyl acrylamide.

In an embodiment of the present invention, the copolymer has a molar ratio of alkyl carboxy ethyl acrylate and alkyl acrylamide in a range of 1:0 to 1:1.

In an embodiment of the present invention, the copolymer has weight average molecular weight (M_{w}) in a range of 5000 to 150000 Daltons.

In another embodiment of the present invention, the pour point depressant polymer improves the cold flow properties of the middle distillate hydrocarbons, wherein the middle distillate hydrocarbons are selected from C₈-C₂₈ hydrocarbons containing n-alkanes or linear alkanes, iso-paraffins, napthenes and aromatics.

In another embodiment, the present invention provides a process for preparing a pour point depressant polymer, the process comprises: mixing the monomers alkyl carboxy ethyl acrylate and alkyl acrylamide in a molar ratio in a range of 1:0 to 1:1 in a solvent at a temperature of 25° C. to 60° C. to obtain a mixture solution; adding 0.1 to 3 wt % of an initiator to the mixture followed by heating at 50° C. to 150° C. for 0.5 to 24 hours for polymerisation to obtain a copolymer solution; cooling the copolymer solution to room temperature followed by addition of methanol to obtain a quencher added copolymer solution; and concentrating the quencher added copolymer solution to obtain the copolymer pour point depressant.

In an embodiment of the present invention, the aromatic solvent is selected from a group consisting of aromatic solvent xylene, toluene, ethyl benzene, C9 aromatic solvent, C10 aromatic solvent, rubber process oil (RPO) and combination thereof.

In an embodiment of the present invention, the initiator is selected from a group consisting of benzoyl peroxide, tertiary butyl hydroperoxide, azobisisobutyronitrile and derivatives thereof.

In an embodiment of the present invention, the quencher is selected from a group consisting of methanol, alcoholic solvents and water.

In an embodiment of the present invention, the monomer is polymerised by free radical polymerisation.

### BRIEF DESCRIPTION OF THE ACCOMPANYING DRAWINGS

These and other features, aspects, and advantages of the present invention will become better understood when the following detailed description is read with reference to the accompanying drawings in which like characters represent like parts throughout the drawings, wherein:

FIG. 1 depicts ¹H NMR spectra of 2-carboxy ethyl acrylate (CEA).

### DETAILED DESCRIPTION OF THE INVENTION

For convenience, before further description of the present disclosure, certain terms employed in the specification, and examples are delineated here. These definitions should be read in light of the remainder of the disclosure and understood as by a person of skill in the art.

The terms used herein have the meanings recognized and known to those of skill in the art, however, for convenience and completeness, particular terms and their meanings are set forth below. The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. The term “at least one” is used to mean one or more and thus includes individual components as well as mixtures/combinations. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps. The term “including” is used to mean “including but not limited to”. “including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

The present disclosure is not to be limited in scope by the specific embodiments described herein, which are intended for the purposes of exemplification only. Functionally equivalent products, compositions, and methods are clearly within the scope of the disclosure, as described herein.

Pour point depressant polymer as disclosed in the present invention acts as a cold flow improver, wherein the cold flow improvers reduce the cold filter plugging point (CFPP), cloud point and pour point of middle distillates.

In the present invention a series of acrylate-acrylamide (R₂CEA-AM) copolymers in different molar ratios were synthesized and used as PPDs for middle distillate hydrocarbons. Reaction conditions were optimized for the synthesis of low molecular weight methacrylate polymers with a suitable degree of polymerization. These polymers are easily soluble in middle distillate hydrocarbons samples. C₁₄CEA-AM (1:0 to 1:1) showed the best suppressive effect compared to the commercial EVA based PPD i.e. pour point of −33° C. at 1000 ppm dosage.

In this aspect, the present invention provides a pour point depressant polymer, wherein the pour point depressant is a poly(alkyl acrylamide)ₘ (alkyl carboxy ethyl acrylate) n copolymer of formula 1:

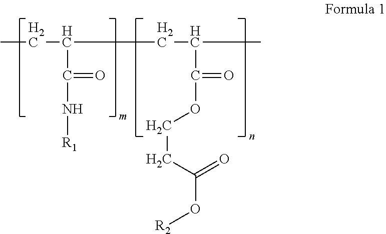

wherein R₁ is selected from H, C1 to C4 alkanes and cyclic alkanes,
wherein R₂ is selected from C₈ to C₂₄ both alkanes and cycloalkanes, preferably alkanes selected from —C₈H₁₇, —C₁₀H₂₁, —C₁₂H₂₅, —C₁₄H₂₉, —C₁₆H₃₃, —C₁₈H₃₇, —C₂₀H₄₁, —C₂₂H₄₅ and —C₂₄H₄₉,
and m is an integer indicating the number of alkyl acrylamide repeating units and n is an integer indicating the number of alkyl carboxy ethyl acrylate repeating units.

In an embodiment of the present invention, the acrylamide monomer is an acrylamide, methyl acrylamide, ethyl acrylamide, propyl acrylamide, butyl acrylamide or acrylamide of cyclic alkane.

In an embodiment of the present invention, the copolymer is prepared by polymerisation of monomers selected from alkyl carboxy ethyl acrylate and alkyl acrylamide.

In another embodiment of the present invention, the copolymer has a molar ratio of alkyl carboxy ethyl acrylate and alkyl acrylamide in a range of 1:0 to 1:1.

In an embodiment of the present invention, the copolymer has a pour point of −33° C. at 1000 ppm dosage.

In an embodiment of the present invention, the copolymer has weight average molecular weight (M_{w}) in a range of 5000 to 150000 Dalton.

In another embodiment of the present invention, the pour point depressant polymer improves the cold flow properties of the middle distillate hydrocarbons, wherein the middle distillate hydrocarbons are selected from C₈-C₂₈ hydrocarbons containing n-alkanes or linear alkanes, iso-paraffins, napthenes and aromatics.

In another aspect, the present invention provides a process for preparing a pour point depressant polymer, the process comprises:
mixing the monomers alkyl carboxy ethyl acrylate and alkyl acrylamide in a molar ratio in a range of 1:0 to 1:1 in a solvent at a temperature of 25° C. to 60° C. to obtain a mixture solution;
adding 0.1 to 3 wt % of an initiator to the mixture followed by heating at 50° C. to 150° C. for 0.5 to 24 hours for polymerisation to obtain a copolymer solution;
cooling the copolymer solution to room temperature followed by addition of quencher to obtain a quencher added copolymer solution; and
concentrating the quencher added copolymer solution to obtain the copolymer pour point depressant.

In an embodiment of the present invention, the aromatic solvent is selected from group consisting of toluene, xylene, ethyl benzene, C9 aromatic solvent, C10 aromatic solvent, rubber process oil and combinations thereof.

In an embodiment of the present invention, the initiator is selected from group consisting of benzoyl peroxide, tertiary butyl hydroperoxide, azobisisobutyronitrile and derivatives thereof.

In an embodiment of the present invention, the quencher is selected from a group consisting of methanol, alcoholic solvents and water.

In an embodiment of the present invention, the alcoholic solvents are methanol, ethanol, propanol and butanol.

In an embodiment of the present invention, the monomer is polymerised by free radical polymerisation.

The efficiency of PAMA-type PPDs polymers could be improved by regulating the ratio between nonpolar moieties and polar moieties, while the arrangement and morphology of wax crystals are altered by long-chain alkyl and polar groups, respectively. The universally used polar groups are maleic anhydride, phenyl, amide, and pyrrolidinone. The unshared electron pairs of the amide group of acrylamides form a conjugated system, which helps the wax crystals to disperse uniformly. To explore the unique properties of acrylamide, a series of alkyl carboxy ethyl methacrylate (RA) acrylamide (AM) copolymers were synthesized using free radical polymerization in different molar ratios.

### EXAMPLES

### Example 1: Synthesis of Monomers & Copolymer

The synthesis routines of alkyl acrylate and copolymers are shown in Scheme. 1. R₂-CEA (R=C₁₂, C₁₄, C₁₆, and C₁₈) was prepared by the reaction between 2-carboxy ethyl acrylate (CEA) and the corresponding alcohol (C₁₂—OH, C₁₄—OH, C₁₆—OH, and C₁₈—OH) at a mole ratio of 1.05:1 at a reaction temperature of 125° C. for 15 hours. Xylene, hydroquinone, and hydrochloric acid were used as solvent, inhibitor, and catalyst, respectively. The reaction products were washed with a 5% NaOH solution two to three times until they became slightly alkaline and then washed with distilled water until they became neutral. Finally, the product was concentrated using a rotary evaporator.

Using xylene as a solvent, R₂-CEA with different alkyl chain lengths (R₂=C₁₂, C₁₄, C₁₆, and C₁₈) and AM were polymerized by free radical polymerization in different molar ratios in the range of 1:0 to 1:1. When the temperature was raised to 50° C., 0.2 wt % of benzoyl peroxide concerning monomers was dissolved in xylene and added dropwise to the reaction system. The mixture is heated for 5 hours at 125° C. The reaction was cooled to room temperature and 0.1 moles of methanol (quencher) was added slowly and the reaction mixture was concentrated using a rotary evaporator, to yield the PPD copolymers.

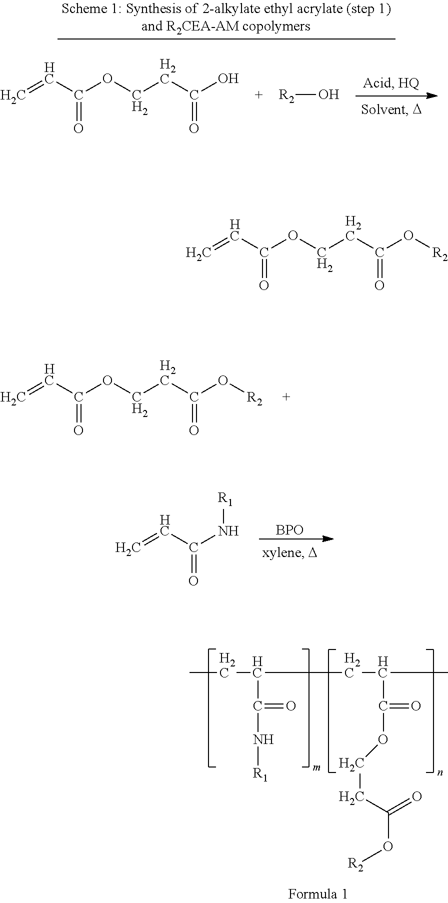

### Characterization

### Proton (¹H) NMR Spectra

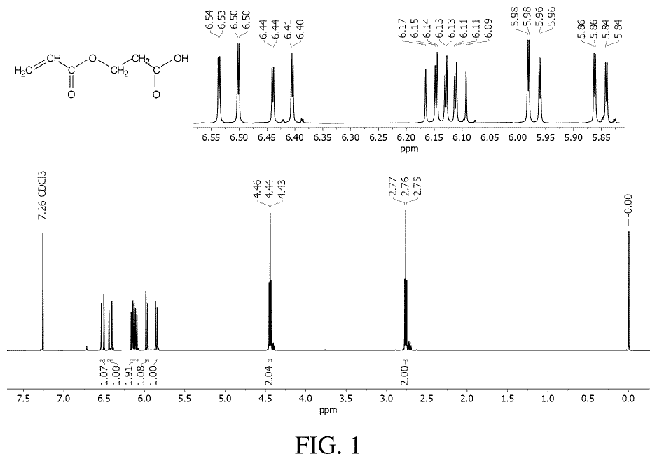

The ¹H NMR of CEA is shown in FIG. 1. In FIG. 1, δ H 6.55 and 5.8 ppm are ascribed to double bond CH and CH₂ protons. The chemical shifts of methylene protons in Cₙ-CEA occur at δ H 4.47 and 2.7 ppm are ascribed to CH₂ protons of ethyl linkage. The chemical shifts of methyl and methylene groups in the Cₙ-CEA monomer occur at 0.85 ppm and 1.27 ppm, δ H 0.88 ppm was assigned to —CH₃ protons, and the chemical shift of the methylene group (—CH₂—) attached to the ester group occurs at 4.15 ppm.

### Gel Permeation Chromatography

The molecular weights (M_{w}) and polydispersity index were determined using Gel Permeation Chromatography (GPC). THF was the mobile phase, and the flow rate was 1 ml/min. Polystyrene is used as a standard material. The weight average molecular weight (M_{w}) of C₁₄CEA-AM copolymer is 13990 Da and the polydispersity index (M_{w}/Mn) is used to measure the molecular weight distribution of PPD. The molecular weights of the synthesized copolymer are within an optimal range and have the optimum applicability as PPDs of diesel fuel.

### N-Alkane Distribution and Pour Point of Pure Diesel Fuel

The carbon number of normal alkanes in diesel fuel is distributed between C₉-C₂₇, and the average carbon number of normal alkanes is 15. The pour point of diesel fuel is −6° C.

### Performance Evaluation for Synthesized Copolymers

The pour point depressive effect of R₂CEA-AM (R₂=C₁₂, C₁₄, C₁₆, and C₁₈) copolymers mainly depends on the alkyl chain length and molar ratios of non-polar (R₂CEA) and polar (AM) monomers. As the ratio of R₂CEA/AM in the polymer increases the number of side chain alkane groups increases, providing a large number of crystallization sites, which changed the growth direction of wax crystals. When the ratio of R₂CEA/AM is continued to increase, the polarity of the polymers became small. Too many alkyl side chains of PPD provide too many crystallization sites, which causes wax crystals to stick to each other and formed a three-dimensional network structure similar to pure diesel. In the present invention, all the copolymers were synthesized by keeping the constant molar ratios between the two monomers in the range of 1:0 to 1:1 (R₂CEA-AM).

All the synthesized copolymers are easily miscible with diesel fuel. The change in the pour point (ΔPP) increases with increasing the dosage of the copolymers. C₁₄CEA-AM (1:0 to 1:1) at 1000 ppm showed the highest pour point reduction compared with other synthesized polymers (C₁₂CEA-AM, C₁₄CEA-AM, C₁₆CEA-AM and C₁₈CEA-AM). In general, PPDs tend to follow the principle of carbon chain matching, which can be evident from these results. The copolymer C₁₄MC-AM (1:0 to 1:1) showed better pour point reduction efficiency compared to benchmark diesel PPD i.e ethylene-vinyl acetate copolymer (EVA). Finally, diesel fuel treated with 1000 ppm of C₁₄CEA-AM (1:0 to 1:1) exerted the lowest pour point of −33° C. Table 1 represents the comparison of change in pour point values of the diesel fuel at different dosages of final formulations i.e. CₓCEA-AM (1:0 to 1:1) (x=12, 14, 16, and 18).

TABLE 1
Performance evaluation of C₁₄CEA-AM (1:0 to 1:1) copolymers with benchmark PPDs

| Dosage (ppm) | | 0 | 50 | 100 | 250 | 500 | 1000 | 2500 |
|---|---|---|---|---|---|---|---|---|
| Cloud point (° C.) | C₁₄CEA-AM copolymer | 0 | 0 | 0 | −1 | −1 | −2 | −1 |
| | benchmark PPD | 0 | 0 | 0 | −1 | −1 | −1 | −1 |
| Pour point (° C.) | C₁₄CEA-AM copolymer | −6 | −12 | −18 | −24 | −30 | −33 | −33 |
| | benchmark PPD | −6 | −9 | −12 | −18 | −21 | −18 | −18 |
| CFPP (° C.) | C₁₄CEA-AM copolymer | 1 | 0 | −1 | −5 | −11 | −14 | −14 |
| | benchmark PPD | 1 | 0 | −1 | −5 | −8 | −9 | −9 |

To further validate the universality of the formulation high pour diesel samples with pour points obtained from diesel treating units DHT and DIU with +12° C. and 0° C. were collected from the HPCL refinery. The results (Table 2) indicate the effectiveness of the formulation even with high pour diesel samples i.e Δ PP of 36° C. for DIU product and 33° C. for DHT product at 1250 ppm dosage.

TABLE 2
Performance evaluation of C₁₄MC-AM (1:0 to
1:1) copolymers with high-pour diesel fuel

| | PPD | Pour point | Pour point |
|---|---|---|---|
| | Dosage | (° C.) | (° C.) |
| Sr. no | (ppm) | DHT-Product | DIU-Product |
| 1 | 0 | 12 | 0 |
| 2 | 1250 | −21 | −36 |
| 3 | 625 | −21 | −30 |
| 4 | 312 | −12 | −24 |
| 5 | 156 | −3 | −15 |
| 6 | 78 | 9 | −9 |

It can be seen from Table 3, that C₁₄CEA-AM (1:0 to 1:1) is highly efficient, and other fuel properties, such as density, kinematic viscosity, flash point, and acid value, with no significant change except PP and CFPP.

TABLE 3
shows the fuel properties of diesel fuel
and diesel fuel treated with 1000 ppm of final
formulation C₁₄MC-AM (1:0 to 1:1). LINK
Excel.Sheet.12 C:\\Users\\31992220\\Desktop\\IVT\\Excel.xlsx
Sheet2!R2C1:R12C4\a\f5\h\*MERGEFORMAT

| | Test | | Diesel + |
|---|---|---|---|
| | method | Diesel | PPD(1000 ppm) |
| Kinematic | D445 | 2.4893 | 2.4933 |
| Viscosity at | | | |
| 40° C. (mm²/s) | | | |
| Density (g/cc) | D4052 | 0.8272 | 0.8272 |
| BS&W (ml) | EN 14103 | 0 | 0.01 |
| Total Acid Number | D664 | 0.063 | 0.095 |
| (mg KOH/g) | | | |
| Water content (ppm) | D4928 | 106 | 90 |
| Oxidation | D7545 | 102.6 g/m3 | 155.1 g/m3 |
| Stability | | | |
| (Minutes) | | | |
| Cloud point | D97 | 0 | −2 |
| (° C.) | | | |
| Pour Point | D97 | −6 | −33 |
| (° C.) | | | |
| CFPP (° C.) | D6371 | 1 | −14 |
| Low Sulfur | D5453 | 6 | 5 |
| (ppm) | | | |
| Copper strip | ASTM | Not worse | Not worse |
| corrosion for | D 130 | than No. 1 | than No. 1 |
| 3 h at 50° C. | | | |
| Carbon | ASTM | 0.2 | 0.2 |
| residue | D 4530 | | |

We claim:

1. A pour point depressant polymer, wherein the pour point depressant polymer is a poly(alkyl acrylamide)ₘ (alkyl carboxy ethyl acrylate), copolymer of formula 1: 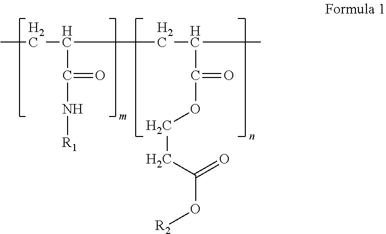
wherein R₁ is selected from the group consisting of H, C1 to C4 alkanes, and cyclic alkanes,
wherein R₂ is selected from the group consisting of C₈ to C₂₄ alkanes, and C₈ to C₂₄ cycloalkanes,
and m is an integer indicating a number of alkyl acrylamide repeating units and n is an integer indicating a number of alkyl carboxy ethyl acrylate repeating units.

2. The pour point depressant polymer as claimed in claim 1, wherein the C₈ to C₂₄ alkanes are selected from the group consisting —C₈H₁₇, —C₁₀H₂₁, —C₁₂H₂₅, —C₁₄H₂₉, —C₁₆H₃₃, —C₁₈H₃₇, —C₂₀H₄₁, —C₂₂H₄₅, and —C₂₄H₄₉.

3. The pour point depressant polymer as claimed in claim 1, wherein the R₂ is selected from the group consisting of C₁₂H₂₅, C₁₄H₂₉, C₁₆H₃₃, and C₁₈H₃₇.

4. The pour point depressant polymer as claimed in claim 3, wherein R2 is C₁₄H₂₉.

5. The pour point depressant polymer as claimed in claim 1, wherein the copolymer comprises monomers of alkyl carboxy ethyl acrylate and alkyl acrylamide.

6. The pour point depressant polymer as claimed in claim 5, wherein the alkyl acrylamide monomer is selected from the group consisting of methyl acrylamide, ethyl acrylamide, propyl acrylamide, butyl acrylamide, and acrylamide of cyclic alkane.

7. The pour point depressant polymer as claimed in claim 1, wherein the copolymer has a molar ratio of alkyl carboxy ethyl acrylate and alkyl acrylamide in a range of 1:0 to 1:1.

8. The pour point depressant polymer as claimed in claim 1, wherein the copolymer has a weight average molecular weight (M_{w}) in a range of 5000 to 150000 Daltons.

9. The pour point depressant polymer as claimed in claim 1, wherein the pour point depressant polymer is characterized to improve cold flow properties of middle distillate hydrocarbons, wherein the middle distillate hydrocarbons are selected from the group consisting of C₈-C₂₈ hydrocarbons containing n-alkanes or linear alkanes, iso-paraffins, napthenes and aromatics.

10. The pour point depressant polymer as claimed in claim 1, wherein the pour point depressant polymer has a pour point of −33° C. at a dosage of 1000 ppm.

11. A process for preparing a pour point depressant polymer, the process comprising:
mixing monomers of alkyl carboxy ethyl acrylate and alkyl acrylamide in an aromatic solvent to obtain a mixture solution;
adding an initiator to the mixture solution followed by heating for polymerisation to obtain a copolymer solution;
cooling the copolymer solution to room temperature followed by addition of a quencher to obtain a quencher added copolymer solution; and
concentrating the quencher added copolymer solution to obtain the pour point depressant polymer.

12. The process as claimed in claim 11, wherein the monomers of alkyl carboxy ethyl acrylate and alkyl acrylamide are mixed in a molar ratio of 1:0 to 1:1.

13. The process as claimed in claim 11, wherein the monomers of alkyl carboxy ethyl acrylate and alkyl acrylamide are mixed in the solvent of at a temperature of 25° C. to 60° C. to obtain the mixture solution.

14. The process as claimed in claim 11, wherein 0.1 to 3 wt % of the initiator is added to the mixture solution.

15. The process as claimed in claim 11, wherein the mixture solution is heated to a temperature in a range of 50° C. to 150° C. for polymerisation to obtain the copolymer solution.

16. The process as claimed in claim 15, wherein the mixture solution is heated for for 0.5 to 24 hours for polymerisation to obtain the copolymer solution.

17. The process as claimed in claim 11, wherein the aromatic solvent is selected from the group consisting of xylene, toluene, ethyl benzene, C9 aromatic solvent, C10 aromatic solvent, rubber process oil, and combinations thereof.

18. The process as claimed in claim 11, wherein the initiator is selected from the group consisting of benzoyl peroxide, tertiary butyl hydroperoxide, azobisisobutyronitrile, and derivatives thereof.

19. The process as claimed in claim 11, wherein the quencher is selected from the group consisting of methanol, alcoholic solvents, and water.

20. The process as claimed in claim 11, wherein the monomers are polymerised by free radical polymerisation.
